(19) United States
(12) Patent Application Publication
(10) Pub. No.: US 2010/0120144 A1
(43) Pub. Date: May 13, 2010

(54) METHOD OF CULTURING CELLS

(75) Inventor: Allan Mishra, Menlo Park, CA (US)

Correspondence Address: KNOBBE MARTENS OLSON & BEAR LLP, 2040 MAIN STREET, FOURTEENTH FLOOR, IRVINE, CA, 92614, US

(73) Assignee: AM Biosolutions, Inc., Menlo Park, CA (US)

(21) Appl. No.: 12/604,270

(22) Filed: Oct. 22, 2009

Related U.S. Application Data
(63) Continuation of application No. 10/581,568, filed on Jun. 2, 2006, now abandoned.
(60) Provisional application No. 60/533,312, filed on Dec. 29, 2003.
(60) Provisional application No. 60/533,350, filed on Dec. 29, 2003.

(51) Int. Cl. C12N 5/071

(52) U.S. Cl. 435/372

(57) ABSTRACT

A blood component such as platelets is concentrated. The concentrate, such as platelet-rich plasma, is used in a cell culture medium to grow and proliferate cells. The cells may be from the same person from which the blood concentrate is obtained. The cells grown in the culture medium may be used to treat a patient which may be the same patient from which the blood was extracted and/or the cells were obtained.

### FIELD OF THE INVENTION

The invention relates generally to the field of cell cultures and more specifically to cell culture media for enhancing cell growth.

### BACKGROUND OF THE INVENTION

Many kinds of cells can be grown in culture, provided that suitable nutrients and other conditions for growth are supplied. Thus, since 1907 when Harrison noticed that nerve tissue explanted from frog embryos into dishes under clotted frog lymph developed axonal processes, scientists have made copious use of cultured tissues and cells from a variety of sources. Such cultures have been used to study genetic, physiological, and other phenomena, as well as to manufacture certain macromolecules using various fermentation techniques known in the art.

In studies of mammalian cell biology, cell cultures derived from lymph nodes, muscle, connective tissue, kidney, dermis and other tissue sources have been used. Generally speaking, the tissue sources that have been most susceptible to the preparation of cell cultures for studies are derivatives of the ancestor mesodermal cells of early development. Tissues that are the progeny of the ancestor endodermal and ectodermal cells have only in recent years become amenable to cell culture, of a limited sort only. The cell types derived from the endoderm and ectoderm of early development include epidermis, hair, nails, brain, nervous system, inner lining of the digestive tract, various glands, and others. Essentially, long-term cultures of normal differentiated human cells, particular certain types of cells, are difficult to obtain. For various types of cartilage cultures see U.S. Pat. No. 5,902,741 issued May 11, 1999.

The cell-types subjected to a procedure of the present invention are derived from various tissues, can be of human origin or that of any other mammal, and may be of any suitable source, such as fibroblast cells, stem cells, cell from a whole pancreas, parotid gland, thyroid gland, parathyroid gland, prostate gland, lachrymal gland, cartilage, kidney, inner ear, liver, parathyroid gland, oral mucosa, sweat gland, hair follicle, adrenal cortex, urethra, and bladder, or portions or multiples thereof.

The tissue is prepared using any suitable method, such as by gently teasing apart the excised tissue or by digestion of excised tissue with collagenase via, for example, perfusion through a duct or simple incubation of, for example, teased tissue in a collagenase-containing buffer of suitable pH and tonic strength. The prepared tissue then is concentrated using suitable methods and materials, such as centrifugation through ficol gradients for concentration (and partial purification). The concentrated tissue then is resuspended into any suitable vessel, such as tissue culture glassware or plasticware. The resuspended material may include whole substructures of the tissue, cells and clusters of cells. For example, such substructures may include fibroblast cells.

The initial culture of resuspended tissue cells is a primary culture. In the initial culturing of the primary culture, the cells attach and spread on the surface of a suitable culture vessel with concomitant cell division. Subsequent to the initial culture, and usually after the realization of a monolayer of cells in the culture vessel, serially propagated secondary and subsequent cultures are prepared by dissociating the cells of the primary culture and diluting the initial culture or its succeeding cultures into fresh culture vessels, a procedure known in the art as passaging. Such passaging results in an expanded culture of cells of the originating tissue. The cell culture is passaged at suitable intervals, such as about once a week or after about two to about three cell divisions of the cultured cells. Longer intervals of two to three weeks or shorter intervals of two to three days would suffice also. For passaging the cell cultures, a dilution of the cultured cells at a ratio of from about 1:2 to about 1:100 is used. Preferably, a ratio of from about 1:4 to about 1:50 is used. More preferably, a ratio of from about 1:4 to about 1:6 is used.

The concentrated prepared tissue, which may be in the form of free cells and/or clumps (where the clumps may constitute ordered substructures of the tissue) is resuspended at any suitable initial cell or presumptive cell density. Suitable cell densities range from about 100 cells to about 1000 cells per square centimeter of surface area of the culture vessel. For useful vessels see U.S. Pat. No. 5,274,084 issued Dec. 21, 1993 and patents and publications cited therein.

Basal media that may be used include those commercially available from Sigma Chemical Co., Life Technologies, Inc., or BioWhittaker Co. Any basal medium may be used provided that at least magnesium ion, calcium ion, zinc ion, bicarbonate ion, potassium ion, and sugar levels can be manipulated to a lower or higher concentration in the resultant medium; in particular, the magnesium ion, calcium ion, bicarbonate ion, and D-glucose levels are required at a lower concentration, zinc ion is required at the same or higher concentration, and potassium ion is required at the same or lower concentration than is usual in standard basal media.

Preferred levels of magnesium ion, as contributed by suitable magnesium salts, such as MgSO₄.7H₂O and MgCl₂.6H₂O, are between 60 and 240 mg/L; more preferred levels of magnesium salts are between 100 and 150 mg/L. Preferred levels of calcium ion, as contributed by suitable calcium salts, such as CaCl₂.2H₂O, are between 25 and 200 mg/L; more preferred levels of calcium ion are between 40 and 125 mg/L. Preferred levels of zinc ion, as contributed by suitable zinc salts, such as ZnSO₄.7H₂O, are between 0.1 and 0.5 mg/L; more preferred levels of zinc ion are between 0.12 and 0.40 mg/L; yet more preferred levels of zinc ion are between 0.15 and 0.20 mg/L. Preferred levels of ascorbic acid are between 30 and 125 mg/L; more preferred levels of ascorbic acid are between 40 and 100 mg/L. Preferred levels of bicarbonate ion, as contributed by suitable bicarbonate salts, such as sodium bicarbonate, are between 175 and 700 mg/L; more preferred levels of bicarbonate ion are between 300 and 400 mg/L. Preferred levels of potassium ion, as contributed by suitable potassium salts, such as potassium chloride, are between 100 and 400 mg/L; preferred levels of potassium ion are between 200 and 325 mg/L; most preferred levels of potassium ion are between 210 and 250 mg/L. Preferred levels of sugar, as contributed by a suitable sugar, such as D-glucose, are between 400 and 1800 mg/L; more preferred levels of sugar are between 600 and 1200 mg/L; most preferred levels of sugar are between 800 and 1000 mg/L. Preferred levels of human placental lactogen are between 3 and 15 μg/ml; more preferred levels of human placental lactogen are between 4 and 13 μg/ml; most preferred levels of human placental lactogen are between 8 and 12 μg/ml. Preferred levels of insulin, as contributed by a suitable naturally-isolated, clonally-derived, or synthesized insulin, such as isolated bovine sodium-insulin, are between 50 and 20,000 ng/ml; more preferred levels of insulin are between 100 and 10,000 ng/ml; most preferred levels of insulin are between 500 and 5,000 ng/ml. (See U.S. Pat. No. 6,008,047 issued Dec. 28, 1999)

The use of animal cell culture for the mass production of cell products such as immunoglobulins, hormones and enzymes is becoming increasingly important from a commercial point of view, and currently there is considerable effort devoted to the development of cell culture techniques for the optimisation of the large scale production of these materials.

Animal cells in culture require a basal nutrient mixture of salts, sugars, amino acids and vitamins. Usually the mixture is supplemented with a biological fluid or extract, in the absence of which most cells lose viability or fail to proliferate. The most commonly used supplement is serum.

The use of supplements, however, can affect the success and reproducibility of a culture. A number of supplement-free media have been described, however, some of which are available commercially [see for example Murakami et al, Proc. Natl. Acad. Sci. USA 79, 1158-1162 (1982); Darfler et al., Exp. Cell Res. 138, 287-295 (1982) and International Patent Specification No. WO 90/03430].

Supplement-free media generally contain a complex mixture of amino acids, salts, vitamins, trace elements, carbohydrates and other growth supporting components such as albumin, insulin, glutamine, transferrin, ferritin and ethanolamine [see for example U.S. Pat. No. 4,816,401]. When cultured in such media, animal cells remain viable for a finite period of time, until one or more essential nutrients in the medium become exhausted. At such time the medium may be supplemented with a feed containing one or more energy sources and one or more amino acids [see for example International Patent Specification No. WO 87/00195]. In this way the culture may be prolonged to increase yield of cells or cell products.

Metal ions, especially ferrous and ferric ions, are essential for animal cell metabolism, and are present in culture media as components of undefined supplements such as serum, or as components of salts and trace elements included in supplement-free media. Cellular demand for metal ions can become high in animal cell culture, especially when high cell densities are reached and in practice this means that metal ions need to be made continuously available in culture to support the growth and viability of cells. To achieve this in a supplement-free medium high concentrations of a simple salt of the metal can be used, but it is often necessary for the metal to be in a chelated form in the medium to facilitate cellular uptake of the metal and/or to avoid the solubility and toxicity problems which can be associated with high metal ion concentrations.

To supply sufficient iron to cells growing in supplement-free media, simple or complex iron salts such as ferrous sulphate, ferric chloride, ferric nitrate or ferric ammonium citrate have been used, where necessary often in combination with a chelating agent. Particular iron chelating agents which have been used in cell culture include the natural proteins transferrin and ferritin; organic acids such as citric acid, iminodiacetic acid and gluconic acid; pyridoxal isonicotinoyl hydrazone; and aurin tricarboxylic acid.

A number of factors are important in selecting an iron chelating agent for general use in supplement-free media for animal cell culture. Thus, the chelating agent must have an appropriate binding affinity for the iron and be able to transport it efficiently across the cell membrane. It must also be cheap, readily available and non-toxic. Increasingly importantly, the chelating agent should be of synthetic, not animal, origin to avoid any possible unwanted contamination of any desired cell product and a consequent increase in the cost of recovery of a pure product. None of the above-mentioned chelating agents meets all of these criteria.

In view of this background and the surrounding business and medical environment the following invention is presented.

### SUMMARY OF THE INVENTION

The invention includes cell culture media and methods for creating cell culture media for the growth and proliferation of all types of cells. The media comprises a concentrate obtained from blood which may be platelet-rich plasma which may be used directly in the creation of the cell culture media or treated, e.g. by methods such as sonification to break open the platelets and obtain a platelet releasate. The blood concentrate enhances cell growth of cells which may be obtained from the same patient as the platelets. Further, the cells grown in the media may be used to treat the same patient from which the blood concentrate and cells were obtained.

A method is disclosed whereby a patient has blood extracted and the patient's blood is used to create a platelet rich plasma (PRR) formulation. The PRP formulation may be buffered to physiological pH (7.4±5%), combined with other components or added directly to a conventional cell culture medium or used to create a cell culture medium of any desired type.

In yet another aspect of the invention fibroblast cells obtained from a patient are grown on a medium comprising PRP and the resulting fibroblasts are formulated and administered to the patient (e.g. the same patient) topically or by injection into and just below the skin.

The invention includes cell culture media and methods for creating cell culture media for the growth and proliferation of follicles in the course of development for maturation of oocytes contained in said follicles, cells of a male germinal line to be matured, oocytes to be fertilized by a spermatozoa, and embryos to be cultured. The media comprises a concentrate obtained from blood which may be platelet-rich plasma which may be used directly in the creation of the cell culture media or treated, e.g. by methods such as sonification to break open the platelets and obtain a platelet releasate. The blood concentrate enhances cell growth of cells which may be obtained from the same patient as the platelets. Further, the cells grown in the media may be used to treat the same patient from which the blood concentrate and cells were obtained and in particular to be reintroduced to the patient during an in vitro fertilization procedure.

The present invention relates to compositions, and methods for increasing the success for in vitro fertilization. More particularly, the present invention relates to compositions, and methods for improving the culturing of embryos. Such compositions, and methods of culturing cells are contemplated for use in any animal system, including humans, and animal husbandry, such as cattle, sheep and swine, and for exotic animals. The following compositions and methods are intended as means to enhance the success of IVF.

A method is disclosed whereby a patient has blood extracted and the patient's blood is used to create a platelet rich plasma (PRR) formulation. The PRP formulation may be buffered to physiological pH (7.4±5%), combined with other components or added directly to a conventional cell culture medium or used to create a cell culture medium of any desired type.

In yet another aspect of the invention follicles in the course of development for maturation of oocytes contained in said follicles, cells of a male germinal line to be matured, oocytes to be fertilized by a spermatozoa, and embryos to be cultured obtained from a patient are grown on a medium comprising PRP and/or platelets releasate and the resulting material is used in the treatment of a patient such as in an in vitro fertilization procedure.

These and other aspects of the invention will become apparent to those skilled in the art upon reading this disclosure.

### BRIEF DESCRIPTION OF THE DRAWINGS

The invention is best understood from the following detailed description when read in conjunction with the accompanying drawing. It is emphasized that, according to common practice, the various features of the drawings are not to-scale. On the contrary, the dimensions of the various features are arbitrarily expanded or reduced for clarity. Included are the following figures:

FIG. 1 is a graph of cell count versus time for cultured fibroblast cells in PRP.

FIG. 2 is a graph of cell count for three different concentrations of PRP releasate and a control.

FIG. 3 is a graph of cell counts over seven days for a control and a culture with sonocated PRP.

FIG. 4 shows twelve photos of the twelve cell cultures under the twelve conditions described in Table 1 after 1 day.

FIG. 5 shows twelve photos of the twelve cell cultures under the twelve conditions described in Table 1 after 3 days. The inserts show cells further to the bottom in the U-shaped wells.

FIG. 6 shows twelve photos of the twelve cell cultures under the twelve conditions described in Table 1 after 7 days.

FIG. 7 shows flow cytometric analysis for the twelve cell cultures under the twelve conditions of Table 1 for CD45RA/CD123 staining of Lin^{neg/low}, CD34^{pos}, CD90^{neg} gated cells. More CMP is present in the presence of the platelet lysate. However, as the 3 KITL, FLT3L, TPO,IL-6 samples show, a certain amount of variability is encountered under these conditions.

FIG. 8 shows flow cytometric analysis of control wells stimulated with defined growth factors at day 8 after plating for the twelve conditions of Table 1.

FIG. 9 shows flow cytometric analysis of wells stimulated with platelet-lysate in the absence of defined growth factors at day 8 after plating for the twelve conditions of Table 1. The well containing 20% platelet lysate was not analyzed by flow cytometry due to the high viscosity of the preparation.

FIG. 10 shows flow cytometric analysis for the twelve cell cultures under the twelve conditions of Table 1 of wells stimulated with defined growth factors combined with platelet lysate at day 8 after plating.

FIG. 11 is a graph of total cell count vs. days after seeding for the results of buffered sonicated trials.

FIG. 12 is a graph as is FIG. 11 where the PRP is buffered but not sonicated.

### DETAILED DESCRIPTION OF THE PREFERRED EMBODIMENT

Before the present compositions, cell culture media and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a platelet” includes a plurality of such platelets and reference to “the carrier” includes reference to one or more carriers and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “platelet” is used here to refer to a blood platelet. A platelet can be described as a minisule protoplasmic disk occurring in vertebrate blood. Platelets play a role in blood clotting. The platelet may be derived from any source including a human blood supply, or the patient's own blood. Thus, the platelets in the composition of the inventions may be autologous. The platelets may be homologous, i.e. form a human but not the same human being treated with the composition.

The term “platelet-rich-plasma,” “PRP” and the like are used interchangeable here to mean a concentration of platelets in a carrier which concentration is above that of platelets normally found in blood. For example, the platelet concentration may be 5 times, 10 times, 100 times or more the normal concentration in blood. The PRP may use the patient's own plasma as the carrier and the platelets may be present in the plasma at a range of from about 200,000 or less to 2,000,000 or more platelets per cubic centimeter. The PRP may be formed from whole blood e.g. by technology disclosed in any of U.S. Pat. Nos. 5,614,106; 5,580,465; 5,258,126 or publication cited in these patents and if needed stored by technology as taught in 2002/0034722A1; U.S. Pat. No. 5,622,867 or publications cited therein. The PRP may comprise blood component other than platelets. It may be 50% or more, 75% or more, 80% or more, 95% or more, 99% or more platelets. The non-platelet components may be plasma, white blood cells and/or any blood component. PRP is formed from the concentration of platelets from whole blood, and may be obtained using autologous, allogenic, or pooled sources of platelets and/or plasma. PRP may be formed from a variety of animal sources, including human sources.

The “dose” of platelets administered to a patient will vary over a wide range based on the age, weight, sex and condition of the patient as well as the patients' own normal platelet concentration, which as indicated above can vary over a ten fold or greater range. Doses of 1 million to 5 million platelets are typical but may be less or greater than such by a factor of two, five, ten or more.

The term “platelet releasate” is the PRP as defined above but treated so that what is inside the platelet shells is allowed to come out. The releasate may be subjected to processing Whereby the platelet shells are removed and/or other blood components are removed, e.g. white blood cells and/or red blood cells or remaining plasma is removed. The pH of the platelet releasate may be adjusted to physiological pH or higher or to about 7.4±10%, 7.4±5%, 7.4±2% or 7.4 to 7.6 as needed.

The terms “treatment”, “treating” and the like are used herein to generally mean obtaining a desired pharmacologic, physiologic or cosmetic effect. The effect may be prophylactic in terms of completely or partially preventing a condition, appearance, disease or symptom thereof and/or may be therapeutic in terms of a partial or complete cure for a condition and/or adverse effect attributable to a condition or disease. “Treatment” as used herein covers any treatment of a condition, disease or undesirable appearance in a mammal, particularly a human, and includes:
(a) preventing the disease (e.g. cancer), condition (pain) or appearance (e.g.

wrinkles) from occurring in a subject which may be predisposed to such but has not yet been observed or diagnosed as having it;
(b) inhibiting the disease, condition or appearance, i.e., causing regression of condition or appearance.
(c) relieving the disease, condition or undesired appearance, i.e., causing regression of condition or appearance.

The invention includes treating patients with cells or components of cells grown on a cell culture media of the invention. For example, fibroblast cells are grown on a media comprising platelet-rich plasma and used in treating older skin to provide a younger appearance, i.e., preventing, inhibiting or relieving the effects of aging on skin and thereby improving the appearance of wrinkled, lined, dry, flaky, aged or photodamaged skin and improving skin thickness, elasticity, flexibility and/or plumpness at one or more particular sites. The cells grown via the present invention may be any type of cells including stems cells which may be embryonic stem cells or adult stem cells, cells from specific organs including but limited to heart, lung, skin, pancrease and liver. The different cells may be obtained from the same patient as the platelets are used to treat the same patient. A range of different therapeutic results can be obtained. For example, heart tissue regrown, skin grafting enhanced, and diabetic patients treated by growing cells which produce insulin. Accordingly, the term “treatment' is intended to mean providing a therapeutically detectable and beneficial effect of any kind on a patient.

The terms “synergistic”, “synergistic effect” and like are used herein to describe improved treatment effects obtained by combining one or more active components together in a composition or in a method of treatment. Although a synergistic effect in some field is meant an effect which is more than additive (e.g., 1+1=3) in the field of treating many diseases an additive (1+1=2) or less than additive (1+1=1.6) effect may be synergistic. For example, if one active ingredient removed 50% of a disease and a second active ingredient removed 50% of the disease the combined (and merely additional) effect would be 100% removal of the disease. However, the effect of both would not be expected to remove 100% of the disease. Often, two active ingredients have no better or even worse results than either component by itself. If an additive effect could be obtained merely by combining treatments than multiple ingredients could be applied to successfully treat any disease and such is not the case.

The term “iontophoresis” means the migration of ionizable molecules through a medium driven by an applied low level electrical potential. This electrically mediated movement of molecules into tissues and in particular into the skin is in addition to the movement obtained via concentration gradient dependent diffusion. If the tissue (e.g. skin) through which the molecules travel also carries a charge, some electro-osmotic flow occurs. However, generally, the rate of migration of molecules with a net negative charge towards the positive electrode and vice versa is determined by the net charge on the moving molecules and the applied electrical potential. The driving force may also be considered as electrostatic repulsion. Iontophoresis usually requires relatively low constant DC current in the range of from about 2-5 mA. For enhancing the delivery of a formulation of the invention such and a platelet releasate through the skin (transdermal iontophoresis), one electrode is positioned over the treatment area and the second electrode is located at a remote site, usually somewhere else on the skin. The return electrode may, for certain applications, be placed elsewhere on the skin as the iontophoretic delivery electrode. With the present invention the return electrode may be similarly positioned on the skin. The applied potential for iontophoresis will depend upon number of factors, such as the electrode configuration and position on the tissue (skin), the nature and charge characteristics of the molecules (e.g. releasate formulation) to be delivered, and the presence of other ionic species within components of the patch and in the tissue extracellular compartments.

As used herein “Collagen” means pharmaceutical grade collagen used in the treatment of human patients. Collagen is a fibrous protein that form fibrils having a very high tensile strength and that has been found in most multicellular organisms. Collagen serves to hold cells and tissues together and to direct the development of mature tissue. Collagen is the major fibrous protein in skin, cartilage, bone, tendon, blood vessels and teeth.

There are many types of collagen which differ from each other to meet the requirements of various tissues. Some examples of types of collagen are as follows: type one [a1(I)]₂ α2 which is found in skin, tendon, bone and cornea; type two [α1(II)₃ which is found in cartilage intervertebral disc, and the vitreous body; type three [α1(III)]₃ which can be found in skin and the cardiovascular system; type four [α1(IV)]₂ α2(IV) which can be found in basement membrane; type five [a1(V)]₂ α2(V) and α1(V)α2(V)α3(V) which is found in the placenta and cornea. Examples of newly identified forms of collagen include: type seven (VII) which is found in anchoring fibrils beneath many epithelial; and types nine (IX), ten (X) and eleven (XI), which are minor constituents of cartilage.

The chemical characterization of native collagen was difficult since its low solubility made isolation of collagen a tedious task. Eventually, it was discovered that collagen from tissues of young animals was not as extensively cross linked as that of mature tissues and thus was more amenable to extraction. For example, the basic structural unit of type I collagen, tropo-collagen, can be extracted in intact form from some young, collagen-containing animal tissues.

Substantial information can be found in patents and publications relating to uses of Collagen. For example, see U.S. Pat. Nos. 4,294,241; 4,668,516; 5,640,941; and 5,716,411 all of which are incorporated herein by reference as are the publications and patents cited in these patents to disclose and describe various ways of using collagen which can in turn be mixed with and administered and used with platelet formulations of the present invention.

### Invention in General

A blood concentrate is obtained from a patient which may be any animal, mammal, or human. The concentrate may be any blood component and may be platelets, platelet-rich plasma (PRP) treated or in its concentrated but native form. The concentrate such as the PRP is used to form a cell culture medium which in turn is used to grow cells. The cells or products such as proteins produced by the cells are used to create a formulation which is administered to a patient to treat the patient. The patient treated may be the same patient from which the blood concentrate and/or the cells are obtained. In addition to cells, tissue such as skin may be cultured on the medium and the tissue used to treat a patient, particularly the patient the tissue was taken from.

In one embodiment of the invention dermal fibroblast cells are obtained from a patient. These cells are cultured in a cell culture medium comprising PRP or platelet releasate obtained from the same patient. The cultured cells are then injected into the same patient to repair subcutaneous dermal tissue, e.g. reduce scars and/or wrinkles. Details regarding certain aspects of this embodiment are described in U.S. Pat. No. 5,591,444 issued Jan. 7, 1997. Also see U.S. Pat. Nos. 6,432,710; 5,858,390; 5,665,372; and 5,660,850 all of which are incorporated herein by reference in their entirety. However, the method of the ‘444 patent is enhanced via the present invention by the use of autologous PRP or releasate to improve growth of the fibroblasts and reduce adverse effects related to exogenous materials.

A cell culture medium of the invention may consist only of platelets, PRP or treated

PRP. However, the medium may be a conventional medium supplemented with platelets, PRP, platelet releasate or combinations thereof. The medium may comprise a cell assimilable source of carbon of carbon, nitrogen, amino acids, iron, inorganic ions, and trace elements.

In an aspect of the method of doing business of the invention cells or tissue are extracted from the patient. These cells or tissue may be of a variety of different types. Blood is then extracted from the same patient and a component of the blood such as platelets of the blood are concentrated to form a concentrate. The extracted cells or tissue are then placed on a cell culture medium which medium is comprised of the concentrate such as the platelets. In one embodiment the platelets are concentrated and subjected to treatment (e.g. sonification) whereby the platelets are caused to break open and provide a releasate. The releasate is used to formulate the culture medium upon which the cells or tissue are cultured. The cells or tissue are maintained on the culture medium under conditions which promote cell growth and proliferation. The cells produced are used to create a formulation. The formulation is administered to the patient to treat a disease. Alternatively, tissue such as skin grown on the medium is used to treat the patient.

In a particular embodiment of the method of the invention eggs are extracted from an adult human female. Blood is extracted from the same human female and the blood is treated in a manner so as to form platelet-rich plasma which is subjected to treatment (e.g. sonification) so as to cause the platelets to open and form a releasate. The extracted egg is fertilized and the fertilized egg placed on a culture medium comprising the releasate. The culture medium containing the patient's own platelet releasate enhances the growth of the fertilized egg. When the fertilized egg reaches an appropriate embryonic state the egg is isolated and placed back into the adult human female which may be the patient from which the egg was extracted.

In general, the business of the invention can involve preparing formulations which are sold to a patient or used on a patient and/or charging the patient for the preparation of formulations. Various methodologies of preparing formulations, growing cells and using formulations to treat patients are described and any of the methodologies can be applied to a method of doing business of the invention.

Formulations of the invention such a formulation comprised of fibroblast cells can be applied topically to and/or injected into and/or under the skin. The formulations comprise platelet and/or fibroblast cells. The platelets and fibroblast cells are preferably obtained from the patient to which the formulation is being administered. A formulation of the invention can be administered to any skin, e.g. to wrinkled, lined, dry, flaky, aged, and photodamaged skin. A range of beneficial results may be obtained, e.g. improving skin thickness, decreasing wrinkles and/or the appearance of wrinkles, improving the elasticity, flexibility and overall appearance.

The examples provided here are of growing human fibroblast cells on a culture medium of the invention. Fibroblast cells produce fibers in connective tissues. Accordingly, such cells are particularly useful in the treatment of skin, e.g. reducing the appearance of wrinkles. However, other types of cells may be produced and such cells formulated to treat a wide range of diseases.

A formulation of the invention may be produced by drawing blood from a human; and centrifuging the blood to obtain a plasma-rich fraction or PRP. The platelet-rich plasma is then combined with a therapeutically acceptable carrier. The formulation is then administered to the patient which may be the same patient from which either or both of the platelets and original cells were obtained.

In an aspect, the invention relates to the method wherein the platelet composition is at or above physiological pH. In an aspect, the invention relates to the method wherein the platelet composition optionally includes platelet releasate. In an aspect, the invention relates to the method further comprising: mixing into the platelet composition one or more of the ingredients selected from thrombin, epinephrine, collagen, calcium salts, pH or adjusting agents. Also useful are materials to promote degranulation or preserve platelets, additional growth factors or growth factor inhibitors, small molecule pharmaceuticals such as NSAIDS, steroids, and anti-infective agents.

In an aspect, the invention relates to the method with the proviso that the platelet composition is substantially free from exogenous activators prior to its administration onto or into the skin.

Media formulations are generally prepared according to methods known in the art. Accordingly, any standard medium, e.g., RMPI-1630 Medium, CMRL Medium, Dulbecco's Modified Eagle Medium (D-MEM), Fischer's Medium, Iscove's Modified Dulbecco's Medium, McCoy's Medium, Minimum Essential Medium, NCTC Medium, and the like can be formulated with PRP or platelet releasate at the desired effective concentration. If desired, media supplements, e.g., salt solutions (e.g., Hank's Balanced Salt Solution or Earle's Balanced Salt Solution), antibiotics, nucleic acids, amino acids, carbohydrates, and vitamins are added according to known methods. If desired, growth factors, colony-stimulating factors, cytokines and the like can also be added to media according to standard methods. For example, media of the invention can contain any of the following substances, alone or in combination, with PRP or platelet releasate: erythropoietin, granulocyte/macrophage colony-stimulating factor (GM-CSF), granulocyte colony-stimulating factor (G-CSF), macrophage colony-stimulating factor (M-CSF), an interleukin (e.g., IL-1, IL-2, IL-3, IL-4, IL-5, etc.), insulin-growth factor (IGF), transferrin, albumin, and stem-cell growth factor (SCF). Media of the invention are useful for culturing a variety of eukaryotic cells, e.g., mammalian cells, yeast cells, amphibian cells, and insect cells. Media can also be used for culturing any tissue or organ. Such media can also be used in a variety of culture conditions and for a variety of biological applications. Examples of such culture conditions include, without limitation, bioreactors (e.g., continuous or hollow fiber bioreactors), cell-suspension cultures, semisolid cultures, liquid cultures, and long-term cell suspension cultures. Media of the invention are also useful for industrial applications, e.g., culturing hybridoma cells, genetically-engineered mammalian cells, tissues or organs.

Cell growth-promoting attributes of PRP and/or platelet releasate is evaluated by any standard assay for analysis of cell proliferation in vitro and in vivo. The art provides animal systems for in vivo testing of cell growth promoting or boosting characteristics of PRP and platelet releasate. Furthermore, a wide variety of in vitro systems are also available for testing growth-promoting or growth-boosting aspects of PRP and/or platelet releasate.

Any cell that proliferates in response to PRP or platelet releasate can be identified according to standard methods known in the art. For example, proliferation of a cell (e.g., a bone marrow cell) can be monitored by culturing in a liquid media containing the test compound, either alone or in combination with other growth factors, added artificially to a serum-free or serum-based medium. Alternatively, such bone marrow cells can be cultured in a semisolid matrix of dilute, agar or methylcellulose, and the test compound, alone or in combination with other growth factors, can be added artificially to a serum-free or serum-reduced medium. In the semisolid matrix the progeny of an isolated precursor cell, proliferating in response to PRP, remain together as a distinguishable colony. For example, a bone marrow cell may be seen to give rise to a clone of a plurality of bone marrow cells, e.g., NK cells. Such culture systems provide a facile way for assaying whether a cell responds to PRP either alone or in combination with other growth factors.

If desired, identification and separation of expanded subpopulations of cells is performed according to standard methods. For example, cells may be analyzed by fluorescence-activated cell sorting (FACS). This procedure generally involves labelling cells with antibodies coupled to a fluorescent dye and separating the labeled cells from the unlabelled cells in a FACS, e.g., FACScan (Becton Dickson). Thus, virtually any cell can be identified and separated, e.g., by analyzing the presence of cell surface antigens (see e.g., Shah et al., J. Immunol. 140:1861, 1988). When a population of cells is obtained, it is then analyzed biochemically or, alternatively, provides a starting population for additional cell culture, allowing the action of the cells to be evaluated under defined conditions in culture.

In a prophetic example, the effect of PRP and/or releasate on the growth of human bone marrow cells may be examined as follows. In general, human bone marrow samples are obtained according to standard procedures after informed consent. For example, bone marrow is obtained from the iliac crest of a healthy donor and the marrow cells are diluted in phosphate-buffered saline at room temperature. Cells are then washed and cultured in an appropriate growth medium. For example, cultures can be set up by inoculating bone marrow cells in 20-30 ml of McCoy's medium containing 50 U/ml penicillin, 50 U/ml streptomycin and 2 mM L-glutamine. Cultures are incubated in the presence or absence of the test compound alone, or in combination with other growth factors, e.g., transferrin or GM-CSF. The cultures are subsequently incubated at 37° C. in a humidified atmosphere containing 5% CO₂, 5% O₂, and 90% N₂ for the desired time period. Cell proliferation assays are performed according to standard methods. For example, replicate samples cultured in the presence and absence of the test compound are analyzed by pulsing the cells with 1-2 micromoles Ci of ³HTdR. After an incubation period, cultures are harvested onto glass-fiber filters and the incorporated ³H measured by liquid scintillation. Comparative studies between treated and control cells, e.g., cell cultured in the presence of PRP versus cells cultured in the absence of PRP, are used to determine the relative efficacy of the test PRP formulation in stimulating cell proliferation. A PRP formulation which stimulates cell proliferation is considered useful in the invention.

### Therapeutic Administration

PRP and/or platelet releasate can be formulated according to known methods to prepare pharmaceutically useful compositions. PRP and/or platelet releasate is preferably administered to the patient in an amount which is effective in preventing or ameliorating the symptoms associated with the disease being treated, e.g. myleotoxcity.

Generally, a dosage comprising 1 to 5 million platelets is adequate. For example, treatment of human patients will be carried out using a therapeutically effective amount of PRP and/or platelet releasate in a physiologically acceptable carrier. Suitable carriers and their formulation are described for example in Remington's Pharmaceutical Sciences by E. W. Martin. The amount of PRP and/or platelet releasate to be administered will vary depending upon the manner of administration, the age, sex, condition and body weight of the patient, and with the type of disease, and size of the patient predisposed to or suffering from the disease.

Routes of administration include, for example, oral, subcutaneous, intravenous, intrperitoneally, intramuscular, transdermal or intradermal injections which provide continuous, sustained levels of the drug in the patient. In other routes of administration, PRP and/or releasate can be given to a patient by injection or implantation of a slow release preparation, for example, in a slowly dissociating polymeric or crystalline form; this sort of sustained administration can follow an initial delivery of the drug by more conventional routes (for example, those described above).

Alternatively, PRP and/or releasate formulations can be administered using an external or implantable infusion pump, thus allowing a precise degree of control over the rate of drug release, or through installation of PRP and/or releasate in the nasal passages or intraplumonary in a similar fashion to that used to promote absorption of insulin, i.e. can be delivered by aerosol deposition of the powder or solution into the lungs.

The therapeutic method(s) and compositions of the present invention may also include co-administration with other human growth factors. Exemplary cytokines or hematopoietins for such use include, without limitation, factors such as an interleukin (e.g., IL-1), GM-CSF, G-CSF, M-CSF, tumor necrosis factor (TNF), transferrin, and erythropoietin. Growth factors like B cell growth factor, B cell differentiation factor, or eosinophil differentiation factors may also prove useful in co-administration with PRP and/or releasate. The dosage recited above would be adjusted to compensate for such additional components in the therapeutic composition. Progress of the treated patient can be monitored by conventional methods.

Treatment is started generally with the diagnosis or suspicion of myelotoxcity and is generally repeated on a regular or daily basis to ameliorate or prevent the progression or exacerbation of the condition. Protection or prevention from the development of a myleotoxcemic condition is also achieved by administration of PRP and/or releasate prior to the onset of the disease. If desired, the efficacy of the treatment or protection regimens is assessed with the methods of monitoring or diagnosing patients for myelotoxcity.

The method(s) of the invention can also be used to treat non-human mammals, for example, domestic pets, or livestock particularly race horses.

### Additional Active Components

Depending on the method of treatment being carried out additional active ingredients can be combined with a formulation of the invention. For example, various anti-cancer compounds could be combined with PRP and/or platelet releasate to treat cancer with the additional active ingredient being chosen based on the compound believed to be the most effective in the treatment of the particular type of cancer being treated. However, in that specific examples provided involve growing fibroblast cells which are particularly useful in the treatment of skin, the following active components are directed to skin treatment.

There are a number of compounds which can have a beneficial effect on treating skin The effect of those components can be enhanced when combined in a composition of the invention. For example, further beneficial results may be obtained by combining the compositions according to the invention with at least one substance chosen from vitamins, particularly the vitamins of group A (retinol) and group C and derivatives thereof such as the esters, especially the palmitates and propionates, tocopherols, xanthines, particularly caffeine or theophylline, retinoids, particularly vitamin A acid, extracts of Centella asiatica, Asiatic and madecassic acids and glycosylated derivatives thereof such as asiasticoside or madecassoside, extracts of Siegesbeckia orientalis, extracts of Commiphora mukl and extracts of Eriobotrya japonica, cosmetically acceptable silicon derivatives such as polysiloxanes, silanols and silicones, C₃-C₁₂ aliphatic alpha-keto acids, particularly pyruvic acid, C₂-C₁₂ aliphatic alpha-hydroxy acids, particularly citric acid, glycolic acid, malic acid and lactic acid, lipoic acid, amino acids, particularly arginine, citrulline and threonine, ceramides, glycoceramides, sphinogosine derivative, particularly type II and III ceramides, phospholipids, forskolin and derivatives thereof, extracts of Coleus, extracts of Tephrosia, elastase inhibitors, particularly ellagic acid and soya peptides, collagenase inhibitors, particularly plant peptides and extracts such as extracts of roots of Coptidis and extracts of roots of Scutellaria baicalensis Georgi, flavonoids such as wogonin, baicalin and abaicalein, aqueous-ethanolic extracts of leaves of Ginkgo biloba, Mosla chinensis, Salvia officinalis and Cinnamommum cassia, catechuic extracts of Camellia sinensis and aqueous extracts of bean shells of Theobroma cacao, anti-inflammatories, particularly phospholipase A2 inhibitors, soothing agents, particularly extracts of liquorice, glycyrrhetinic acid and ammonium glycyrrhizinate, hydrating agents, particularly polypols, propylene glycol, butylenes glycol, glycerol and hyaluronic acid, agents for combating stretch marks, particularly extracts of horse chestnut and escin, agents for protecting or improving the microcirculation, particularly bioflavonoids from Ginkgo biloba, isodon, extracts if Ami visnaga, visnadine and ruscogenin, free radical inhibitors, particularly polyphenols such as PCO (procyanidolic oligomers) and derivatives thereof and plant extracts, particularly extracts of Curuma longa, antiseborrhea agents, such as a 5-alpha-reductase inhibitor, particularly an extract of Pygeum africanum, and stimulants of the microcirculation of the blood, such as cepharanthine and methyl nicotinate.

The compositions according to the invention can advantageously contain substances for protecting the skin from the harmful effects of the sun, such as solar filters, individually or in combination, especially UV A filters and UV B filters, particularly titanium oxides and zinc oxides, oxybenzone, Parsol MCX, Parsol 1789 and filters of vegetable origin, substances for limiting the damage caused to the DNA, particularly those for limiting the formation of thymine dimmers, such as ascorbic acid and derivatives thereof and/or Photonyl®, and substances for contributing to the elimination of liver spots, such as inhibitors of melamin or tyrosinase synthesis.

The invention also relates to the method further comprising: mixing into the platelet composition substantially simultaneously with its topical application to the skin, with one or more of the ingredients selected from thrombin, epinephrine, collagen, calcium salts, and pH adjusting agents. Also useful are materials to promote degranulation or preserve platelets, additional growth factors or growth factor inhibitors, small molecule pharmaceuticals such as NSAIDS, steroids, and anti-infective agents.

In yet another aspect, the invention relates to a dermatological composition comprising: platelet releasate wherein the composition is at a pH greater than or equal to physiological pH, and wherein the composition comprises substantially no unactivated platelets.

PRP is a concentration of platelets greater than the peripheral blood concentration suspended in a solution of plasma, with typical platelet counts ranging from 500,000 to 1,200,000 per cubic millimeter, or even more. PRP is formed from the concentration of platelets from whole blood, and may be obtained using autologous, allogenic, or pooled sources of platelets and/or plasma. PRP may be formed from a variety of animal sources, including human sources.

Platelets are cytoplasmic portions of marrow megakaryocytes. They have no nucleus for replication; the expected lifetime of a platelet is some five to nine days. Platelets are involved in the hemostatic process and release several initiators of the coagulation cascade. Platelets also release cytokines involved with initiating wound healing. The cytokines are stored in alpha granules in platelets. In response to platelet to platelet aggregation or platelet to connective tissue contact, as would be expected in injury or surgery, the cell membrane of the platelet is “activated” to secrete the contents of the alpha granules. The alpha granules release cytokines via active secretion through the platelet cell membrane as histones and carbohydrate side chains are added to the protein backbone to form the complete cytokine. Platelet disruption or fragmentation, therefore, does not result in release of the complete cytokine.

A wide variety of cytokines are released by activated platelets. Platelet derived growth factor (PDGF), transforming growth factor-beta (TGF-b), platelet-derived angiogenesis factor (PDAF) and platelet derived endothelial cell growth factor (PD-ECGF) and insulin-like growth factor (IGF) are among the cytokines released by degranulating platelets. These cytokines serve a number of different functions in the healing process, including helping to stimulate cell division at an injury site. They also work as powerful chemotactic factors for mesenchymal cells, monocytes and fibroblasts, among others.

Historically, PRP has been used to form a fibrin tissue adhesive through activation of the PRP using thrombin and calcium, as disclosed in U.S. Pat. No. 5,165,938 to Knighton, and U.S. Pat. No. 5,599,558 to Gordinier et al., incorporated in their entirety by reference herein. Activation results in release of the various cytokines and also creates a clotting reaction within various constituents of the plasma fraction. The clotting reaction rapidly forms a platelet gel (PG) which can be applied to various wound surfaces for purposes of hemostasis, sealing, and adhesion.

In another embodiment, the inventive platelet composition may comprise releasate from platelets, in addition to platelets themselves. The releasate comprises the various cytokines released by degranulating platelets upon activation. Many activators of platelets exist; these include calcium ions, thrombin, collagen, epinephrine, and adenosine diphosphate. Releasates according to the invention may be prepared according to conventional methods, including those methods described in U.S. Pat. No. 5,165,938 to Knighton, and U.S. Pat. No. 5,599,558 to Gordinier et al. The releasates alone or in a dermatologically acceptable carrier may be topically applied and/or injected into the skin.

One disadvantage of conventional releasate strategies associated with the use of PRP as PG is the use of thrombin as a preferred activator. In particular, much thrombin used in PG is bovine thrombin, which can create problems due to contamination issues regarding prions which cause Creutzfeldt-Jakob disease. Many bovine materials are suspect due to possible prion contamination, and so use of bovine thrombin is disfavored. Human pooled thrombin is likewise disfavored due to the potential of contamination with various infectious agents such as viruses, prions, bacteria and the like. Recombinant human thrombin might also be used, but may be expensive. Any of the platelets, fibroblast cells, thromin, or formulations of the invention or components thereof may be tested for the presence of prions using assays known in the art such as disclosed in U.S. Pat. No. 6,620,629 issued Sep. 16, 2003 and; U.S. Pat. Nos. 6,221,614; 6,617,119 issued Sep. 9, 2003; and U.S. Pat. No. 5,891,641.

It is a particular advantage of the present invention that exogenous or extra activators need not be administered to a patient. Collagen, a major component of connective tissues, is a strong activator of platelets. Thus, when the inventive platelet composition is administered to skin, platelets in the platelet composition may bind to the collagen and then be activated. This reduces or eliminates the need for administering an exogenous activator such as thrombin. The disadvantages of thrombin use have been noted above. Other strong activators, such as calcium ions, can cause severe pain, unintentional clotting, and other undesirable side effects. Thus, in an embodiment of the invention, no or substantially no exogenous activator is present or added as part of the inventive platelet composition, or is used in the preparation of the inventive platelet composition. Of course, exogenous activators may still be employed if a physician determines that they are medically necessary or desirable. Thus, the composition of the invention may consist only of platelets as the active ingredient.

The platelet composition may be prepared using any conventional method of isolating platelets from whole blood or platelet-containing blood fractions. These include centrifugal methods, filtration, affinity columns, and the like. If the platelet composition comprises PRP, then conventional methods of obtaining PRP, such as those disclosed in U.S. Pat. Nos. 5,585,007 and 5,788,662 both to Antanavich et al., incorporated herein by reference in their entirety, may be utilized.

Adjusting the pH of platelet compositions has been used to prolong the storage time of unactivated platelets, as disclosed in U.S. Pat. No. 5,147,776 to Koerner, Jr. and U.S. Pat. No. 5,474,891 to Murphy, incorporated by reference herein. pH may be adjusted using a variety of pH adjusting agents, which are preferably physiologically tolerated buffers, but may also include other agents that modify PRP pH including agents that modify lactic acid production by stored platelets. Especially useful are those pH adjusting agents that result in the pH of the platelet composition becoming greater than or equal to physiological pH. In an embodiment, the pH adjustment agent comprises sodium bicarbonate. Physiological pH, for the purposes of this invention, may be defined as being a pH ranging from about 7.35 to about 7.45. pH adjusting agents useful in the practice of this invention include bicarbonate buffers (such as sodium bicarbonate), calcium gluconate, choline chloride, dextrose (d-glucose), ethylenebis(oxyethylenenitrilo)tetraacetic acid (EGTA), 4-(2-hydroxyethyl)-1-piperazineethanesulfonic acid (HEPES), maleic acid, 4-morpholinepropanesulfonic acid (MOPS), 1,4-piperazinebis(ethanesulfonic acid) (PIPES), sucrose, N-tris(hydroxymethyl)methyl-2-aminoethanesulfonic acid (TES), tris(hydroxymethyl)aminomethane (TRIS BASE), tris(hydroxymethyl)aminomethane hydrochloride (TRIS.HCl), and urea. In a preferable embodiment, the pH adjusting agent is a bicarbonate buffer, more preferably, sodium bicarbonate.

### Cell Cultures

The cell cultures of the present invention involved the use of PRP and, for example may use PRP from the same patient the cells (e.g. fibroblast cells) being cultured were obtained from.

Example 5 below shows the cell culture with PRP therein and Example 6 shows the cell culture with three different concentrations of platelet releasate therein. The platelets may be treated in any manner to open the platelets or allow the releasate to escape. The treatment may be with an energy wave (e.g. ultra sound), agitation, temperature (heating/cooling-freezing/thawing), and chemical treatments or any combination thereof.

The cells such as fibroblasts and keratinocytes used in accordance with the present invention may be either autogenic or allogenic relative to the platelets and/or the patient treated with the cells grown. The use of allogenic cells enables the production and storage of the living skin equivalent of the present invention thereby avoiding delays in procuring grafts for the treatment of wounds. Both cell types, keratinocytes and fibroblasts could be stored frozen for months as single cell suspensions, using published methods. After thawing these cells should maintain their viability and grow readily in culture. (See U.S. Pat. No. 6,039,760 issued Mar. 21, 2000)

### Topical Formulations

The PRP and fibroblast cells obtained can be dispersed in, mixed with or combined in any fashion with a dermatologically acceptable carrier to create a topical formulation. The formulation may be an ointment, cream, lotion, oil or the like that can be placed on the skin of a human. The carrier may be comprised of natural, refined or synthetic oils or combinations thereof. The carrier may be derived from a liquid petroleum gelled by the addition of a polyethylene resin. Composition based on animal fats, and/or vegetable oils may be used including lard, benzoinated lard, olive oil, cottonseed oil and the like. Examples of topical formulations are described and disclosed in publications such as Remington's Pharmaceutical Sciences, (18^{th} Ed.) Mack Publishing, Co. 1990. Such formulations may comprise a preservative and bacterialcidal and/or bacterialstatic compounds as well as perfumes and coloring agents.

The topical formulations may have a buffer adder to the PRP or have the buffer in the carrier. The pH of the formulation should be balanced to obtain a pH close to physiological pH e.g. about 7.4±10% or ±5%, or 7.2 to 7.6.

The presence of other active ingredients may require a different overall pH for the formulation as some active ingredients require a particular pH range. The releasate, platelets and/or the platelets and releasate may be combined with the carrier over a wide range of concentrations, e.g. 1%, 10%, 25%, 50%, 75%, 90%, 95%, 99% carrier with the remainder being PRP, platelets, platelet releaseate or combinations thereof with or without an additional active ingredient.

### Injectable Formulations

Injectable formulations may be comprised of PRP, or platelet releasate, water and buffer to balance the pH to near physiological pH e.g. about 7.4±10%, 7.4±5% or 7.2 to 7.6. Suitable formulations of the invention may be prepared using technology as taught within Remington's cited above.

Both injectable and topical formulations may further comprise fibroblast cells particularly as cultured per the present invention. Both injectable and topical formulations may further comprise PRP releasate and/or other pharmacologically active components.

The culture medium of the invention may be used to create a wide range of different types of cells. Once grown on a cell culture medium of the invention the cells or products produced from the cells can be formulated into a pharmaceutically acceptable formulation and administered to a patient which may be a human patient and may be the same human patient from which the platelets and/or the cells were derived.

### Examples

The following examples are put forth so as to provide those of ordinary skill in the art with a complete disclosure and description of how to make and use the present invention, and are not intended to limit the scope of what the inventor regard as his invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

PRP was prepared using a centrifuge unit made by Harvest (Plymouth, Mass.). (Similar units are available as The Biomet GPS system, the Depuy Symphony machine and the Medtronic Magellan machine.) Approximately 55 cc of blood was drawn from the patient using a standard sterile syringe, combined with 5 cc of a citrate dextrose solution for anticoagulation, and then spun down to isolate the platelets according to the manufacturer's protocol. These platelets were then resuspended in approximately 3 cc of plasma. The resulting platelet rich plasma solution (PRP) was quite acidic and was neutralized with using approximately 0.05 cc of an 8.4% sodium bicarbonate buffer per cc of PRP under sterile conditions to approximately physiologic pH of 7.4. The PRP was not activated through addition of exogenous activators. This PRP composition is referred to herein as autologous platelet extract (APEX).

### Example 2

Fifty cc of whole blood is drawn from a patient, and then prepared according to the method of Knighton, U.S. Pat. No. 5,165,938, column 3. The PRP is activated according to Knighton using recombinant human thombin. The degranulated platelets are spun down and the releasate containing supernatant is recovered. The releasate may be optionally pH adjusted to a pH of 7.4 using sodium bicarbonate buffer.

### Example 3

Thirty ml of whole blood were drawn from a patient. A platelet composition was prepared according to Example 1 of U.S. Pat. No. 5,510,102 to Cochrum, incorporated herein by reference in its entirety, except that no alginate is added to the platelet composition.

### Example 4

### Cell Cultures of Any Tissue

A researcher or clinician wishes to grow a cell culture of either fibroblasts or osteoarthritic cartilage cells. Using the technique of Example 1, an autologous platelet extract (APEX) is obtained and buffered to physiologic pH.

The cells are then isolated and grown in a media rich in the APEX in various conditions and dilutions. The APEX promotes cell differentiation and production of proteins such as collagen. The APEX may augment or promote the ability of the cells to transform into normal cells. Without intending to be limited by theory, it is hypothesized the APEX may convert the osteoarthritic cartilage cells to a more functional cell line that is reinjected into a diseased or injured joint. Alternatively, the APEX is directly introduced into an osteoarthritic joint to reverse the course of the disease. This is done under local anesthesia in a sterile manner.

### Example 5

### Human Fibroblast Proliferation in Buffered Platelet Rich Plasma

Platelet rich plasma has been used to augment bone grafting and to help accelerate or initiate wound healing. Fibroblasts are important components of the wound healing process. This example shows that human fibroblast cells will proliferate more in fetal bovine serum that has been augmented with a proprietary formulation of buffered platelet rich plasma.

Human fibroblasts were isolated and then put into culture with 10% fetal bovine serum that had been augmented with a proprietary formulation of buffered platelet rich plasma (Group 1) or in 10% fetal bovine serum alone (Group 2). Initial cell counts were 25,000 in both groups.

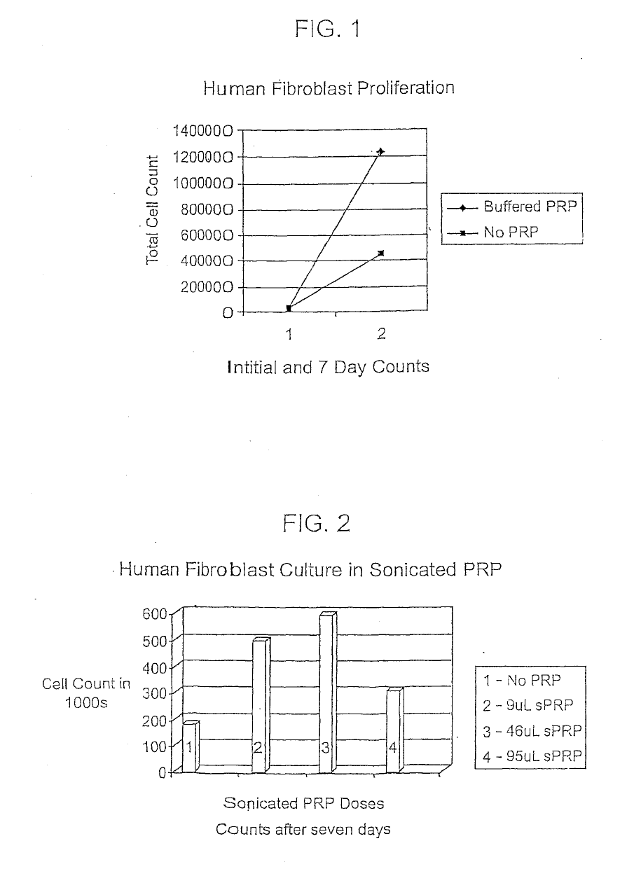

Seven days after initiating the culture experiment, the cells in each group were counted. The average total cell count in Group 1 (buffered PRP added) was 1,235,000. The average total cell count in Group 2 (No PRP) was 443,000. The group that was augmented with the buffered platelet rich plasma of the invention had 2.8 times the proliferation of the control group at seven days. (See FIG. 1)

Buffered platelet rich plasma augments human fibroblast proliferation when compared to the use of fetal bovine serum alone. This has significant implications for the use of buffered platelet rich plasma for either acute or chronic wound healing.

### Example 6

### Human Fibroblast Proliferation in Sonicated Platelet Rich Plasma

Human fibroblasts were isolated and then put into four different cultures. Three of the cultures comprised 10% fetal bovine serum that had been augmented with 9 uL, 46 uL, and 95 uL of buffered and sonicated platelet rich plasma. The fourth served as the control and was comprised of 10% fetal bovine serum Initial cell counts were 20,000 in both groups. Variable doses of the sonicated PRP (sPRP) were seeded with cells.

Four days after initiating the culture experiment, the cells in each of the four groups were counted and the results are shown in FIG. 2. The cell count in the control group (No PRP) was 180,000 cells. The cell counts in the sonicated PRP group were as follows: 496,000 (9 uL dose of sPRP), 592,000 (46uL dose of sPRP) and 303,000 (95uL dose of sPRP).

This experiment shows that buffered, and sonicated platelet rich plasma augments human fibroblast proliferation when compared to the use of fetal bovine serum alone.

### Example 7

### Human Fibroblast Proliferation in Sonicated Platelet Rich Plasma

Human fibroblasts were isolated and then put into two different cultures. One of the cultures comprised 10% fetal bovine serum that had been augmented with buffered and sonicated platelet rich plasma. The other served as the control and was comprised of 10% fetal bovine serum. Initial cell counts were 20,000 in both groups.

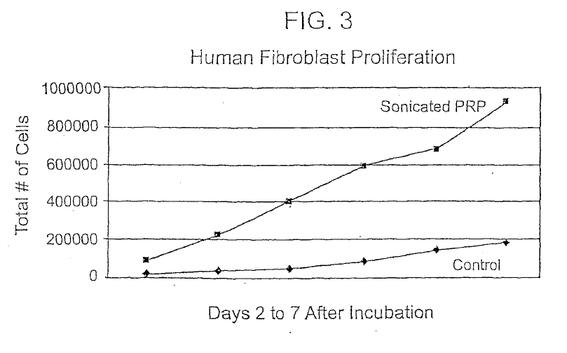

Seven days after initiating the culture experiment, the cells in each of the two groups were counted and the results are shown in FIG. 3. The cell count in the control group (No PRP) was 183,600 cells. The cell count in the sonicated PRP group was 924,800 cells.

This experiment shows that buffered, and sonicated platelet rich plasma augments human fibroblast proliferation when compared to the use of fetal bovine serum alone. These results show the ability of the platelet releasate to promote cell growth and in particular fibroblast cells which are essential to firm, young looking skin.

### Examples 1-7

### Human Fibroblast Proliferation

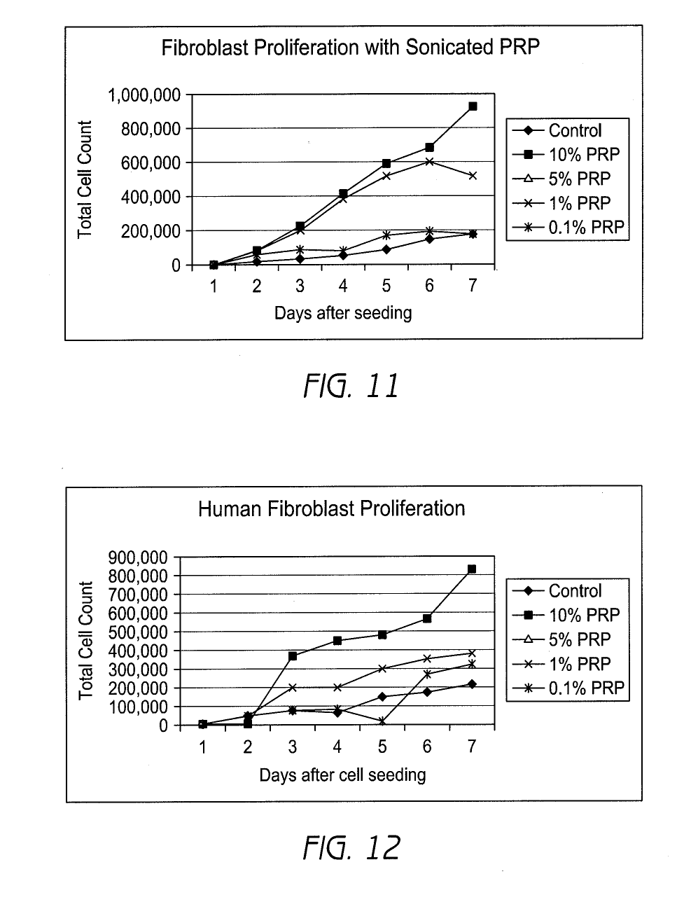

Using methods and procedures as described in Examples 1-7 data was obtained for both a sonicated trial and an unsonicated trial in five different cell cultures. Each group of five cultures is seeded with 10,000 cells and include a control culture where PRP was compared against test cultures at 0.1% PRP, 1% PRP, 5% PRP and 10% PRP. The results are shown below in the two tables and are graphed in FIGS. 11 and 12.

Buffered and SONICATED Platelet Rich Plasma vs. Control as a culture media
Fibroblast Count

| Well Media | Initial Seed | Day 2 | Day 3 | Day 4 | Day 5 | Day 6 | Day 7 | Versus Control |
|---|---|---|---|---|---|---|---|---|
| Control | 10,000 | 17,600 | 36000 | 50700 | 86700 | 146200 | 183600 | 1.0x |
| 10% PRP | 10,000 | 85250 | 224250 | 411450 | 588200 | 680000 | 924800 | 5.04x |
| 5% PRP | 10,000 | 73150 | 175500 | 352950 | 433500 | 510000 | 363800 | 1.98x |
| 1% PRP | 10,000 | 79200 | 199875 | 382200 | 510850 | 595000 | 519350 | 2.83x |
| 0.1% PRP | 10,000 | 58100 | 89250 | 82550 | 169100 | 198900 | 175100 | 0.95x |

The results with the sonicated PRP was repeated where the PRP releasate was frozen (freezing may be at −70° C.) and thawed and thereafter used in cell cultures where similar results were obtained.

Human Fibroblast Proliferation
Buffered Platelet Rich Plasma vs. Control as a culture media
Fibroblast Count

| Well Media | Initial Seed | Day 2 | Day 3 | Day 4 | Day 5 | Day 6 | Day 7 | Versus Control |
|---|---|---|---|---|---|---|---|---|
| Control | 10,000 | 48,500 | 81750 | 65800 | 150300 | 176800 | 214200 | 1.0x |
| 10% PRP | 10,000 | 11880 | 371250 | 448700 | 482600 | 567800 | 824500 | 3.85x |
| 5% PRP | 10,000 | 66550 | 108000 | 113100 | 258660 | 304300 | 380800 | 1.78x |
| 1% PRP | 10,000 | 50600 | 202500 | 198575 | 299840 | 352750 | 382500 | 1.79xx |
| 0.1% PRP | 10,000 | 54450 | 82500 | 82550 | 22830 | 268600 | 323000 | 1.51x |

### Examples 8-18

### Platelet Extract Using Therapeutic Culture Conditions for FACS-Purified Human HSC

Experiments were carried out to determine whether the growth factor rich, but only partially defined, platelet lysates obtained by sonification are beneficial in culturing human HSC with the aim of (i) expanding the number of HSC present in the culture or (ii) expanding the number of myeloid progenitors present in the culture.

Cells: CD34 enriched cells from G-CSF mobilized peripheral blood from healthy volunteers were stained with Lin, CD34 and CD90. Lin^{neg}, CD34^{pos} CD90^{pos} cells were using a FACSaria and deposited at 500 cells per well in 96 wells u-bottom plates.

Culture conditions: medium Xvivo 15 supplemented with 2-mercaptoethanol, penicillin and streptomycin and growth factors. The base growth factor mix used was KITL (100 ng/ml), FLT3L (100 ng/ml) and TPO (50 ng/ml). In addition to these factors IL-6 and IL-3 were used at 10 ng/ml. The platelet lysate was used at 1%, 5% and 20% of the final volume.

The cells were sorted and deposited in the wells. Platelets were sonicated to obtain lysate which was combined with concentrated growth factors, added to the cells and incubated at 37° C. in a fully humidified incubator at 5% CO₂. The cultures were then transferred to incubators.

The conditions tested for each of Examples 8-19 are shown below in Table 1. (one well with 500 HSC per condition):

TABLE 1

| No GF, no PL | No GF, 1% PL | No GF, 5% PL | No GF, 20% PL |
|---|---|---|---|
| KITL, FLT3L, | KITL, FLT3L, | KITL, FLT3L, | KITL, FLT3L, TPO + |
| TPO, noPL | TPO + 1% PL | TPO + 5% PL | 20% PL |
| KITL, FLT3L, | KITL, FLT3L, | KITL, FLT3L, | KITL, FLT3L, TPO, |
| TPO, IL-6, | TPO, IL-6 + | TPO, IL-6 + | IL-6 + 20% PL |
| no PL | 1% PL | 5% PL | |

In addition to these conditions several wells were plated with KITL, FLT3L and TPO, KITL, FLT3L, TPO and IL-3 and KITL, FLT3L, TPO and IL-6.

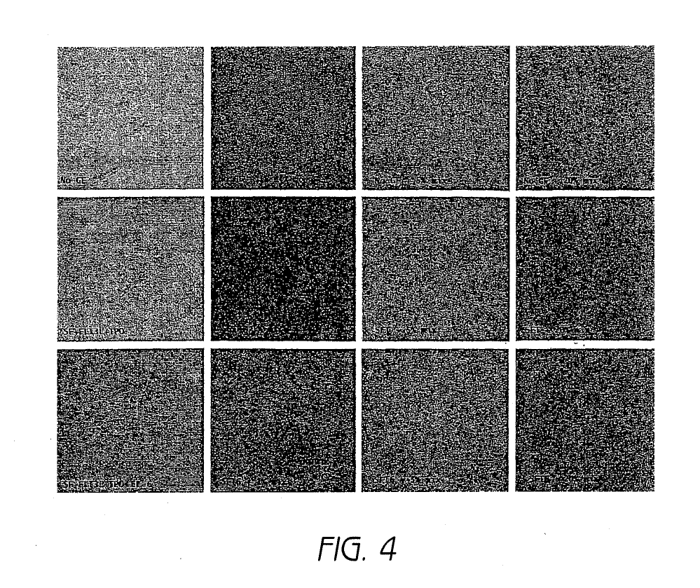

Analysis. Photographs of the wells were taken after approx. I, 4 and 7 days of culturing (FIGS. 4, 5 and 6).

For the twelve cell cultures shown in FIG. 4, no live cells are left in the well that received neither growth factor nor platelet mix. Living and proliferating HSC (derived cells) can readily be seen at day one in the wells supplemented with growth factors. The HSC are obscured in the wells supplemented with platelet lysate by the unlysed red blood cells present in these wells. Nevertheless it is clear that HSC derived cells are present in the wells receiving both growth factors and 1-5% of the platelet lysate.

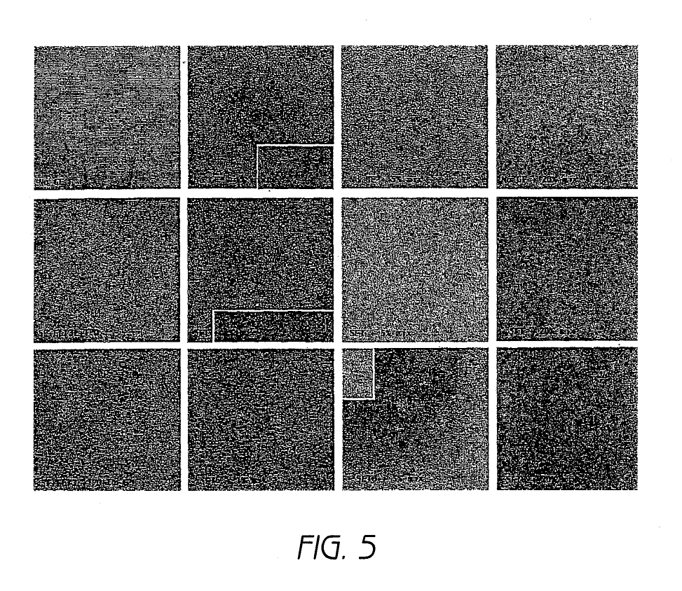

FIG. 5 Day 3. Continued rapid proliferation has resulted in sizable colonies in the wells receiving only growth factors. IL-6 clearly augments the growth by KITL+TPO+FLT3L. HSC-derived cells can also be seen in the wells containing 1 and 5% platelet lysate, although it is difficult to assess numbers due to the red cells present. The wells that contain 20% platelet lysate have a high viscosity and are nearly opaque. Few details can be seen. The insets show cells further to the side in the u-bottom wells.

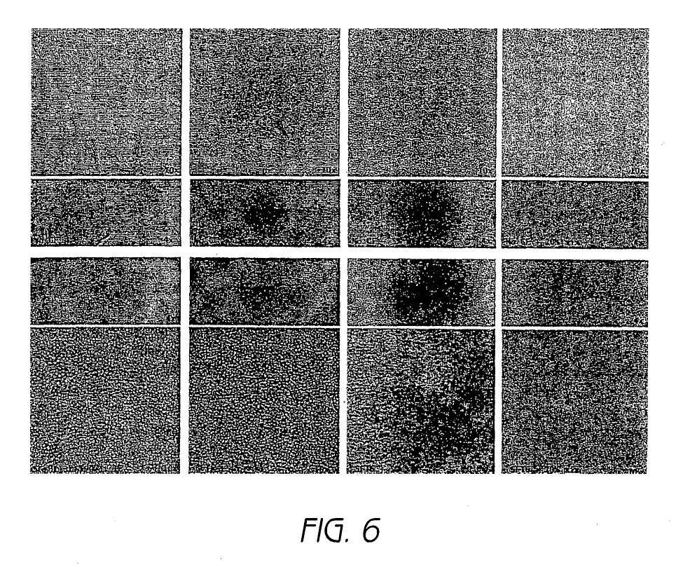

FIG. 6 Day 7. These photographs were taken post-harvest and flow cytometric analysis of the middle row of wells. Micrographs at a lower magnification are included to better illustrate the relative sizes of the colonies present. HSC-derived cells can be seen in the well supplemented with 1% platelet lysate without additional factors (in addition to lysate-derived red blood cells). It is difficult to see whether this is the case in the well supplemented with 20% lysate, and it is impossible to see any details in the well supplemented with 20% lysate. Continued rapid proliferation is seen in the wells supplemented with KITL, FLT3L and TPO, and this does not seem to be affected (in a negative fashion) by the platelet lysate.

The cells from the middle rows (which received KITL, FLT3L and TPO as the base mix) were harvested at day 5 and analyzed by flow cytometry.

The cells were compared with wells receiving IL-6 or IL-3 at 10 ng/ml. Absolute cell numbers in the wells were determined by mixing in a known quantity of fluorescent beads.

The remaining wells were harvested after 8 days of culture and analyzed in a similar fashion. The antibodies used in the analysis include CD45RA^{FITC}, CD123^{PE}, Lin^{Cy5PE} and CD34^{APC}.

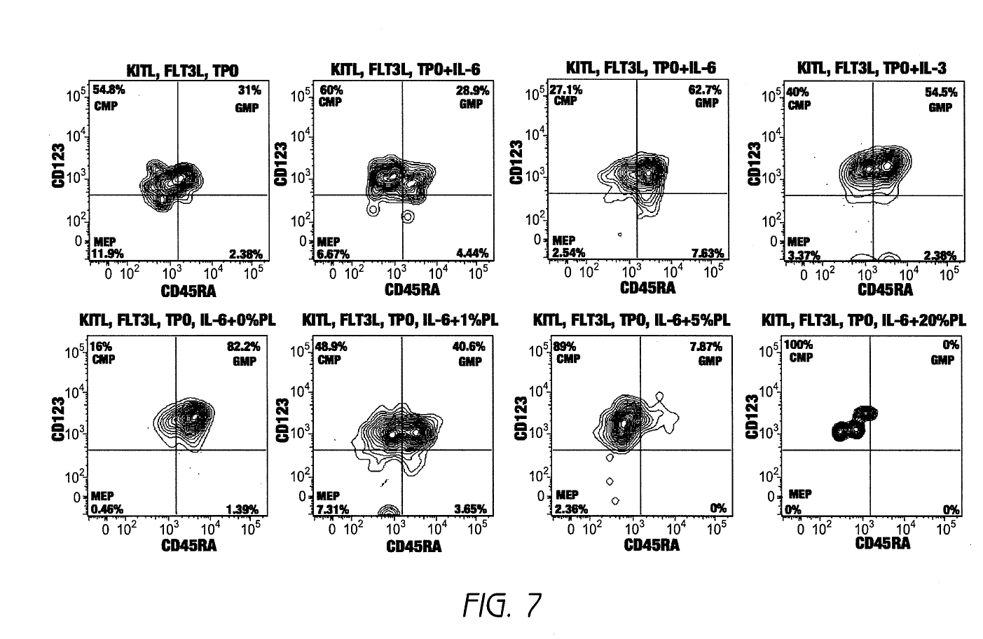

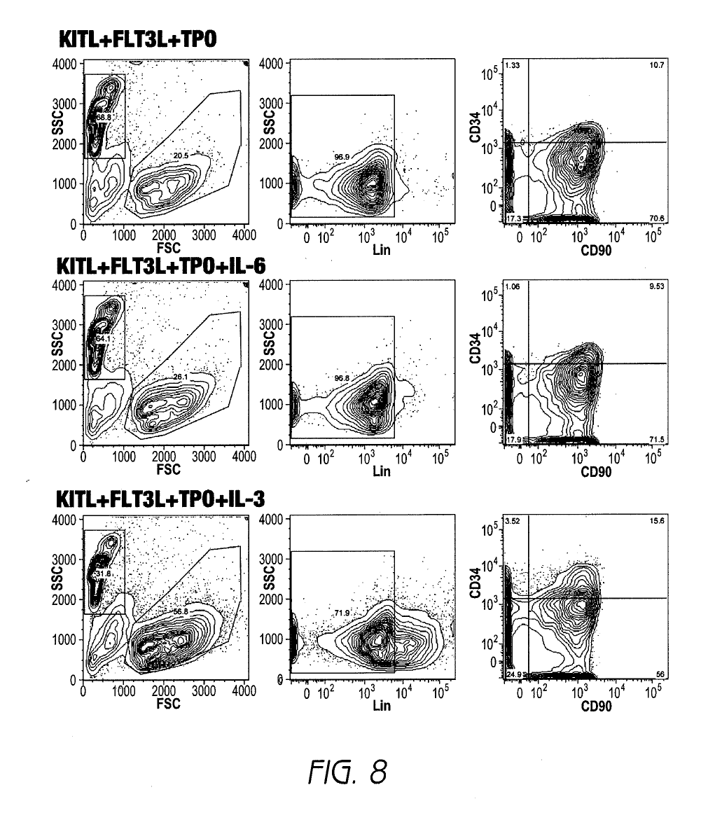

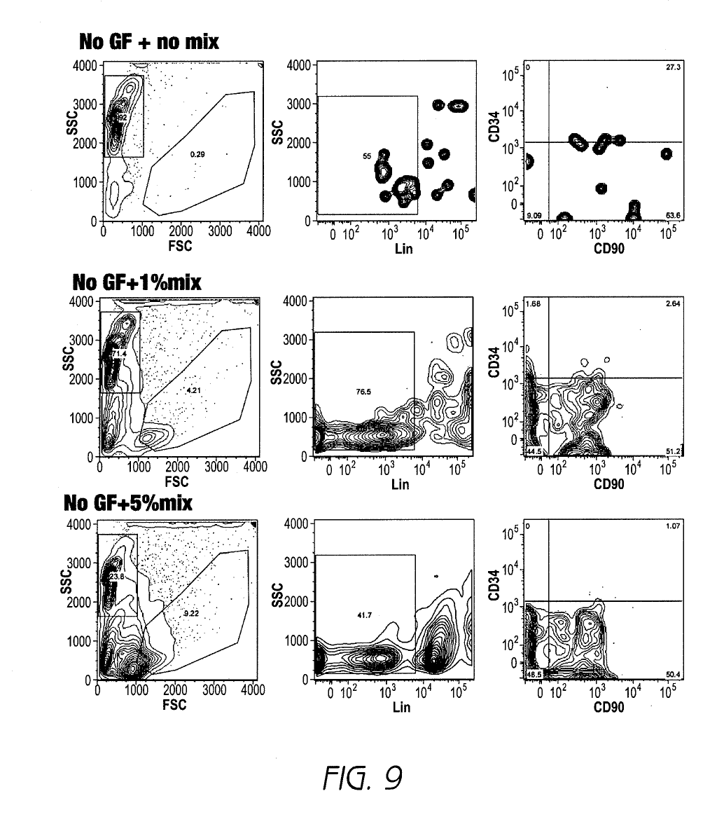

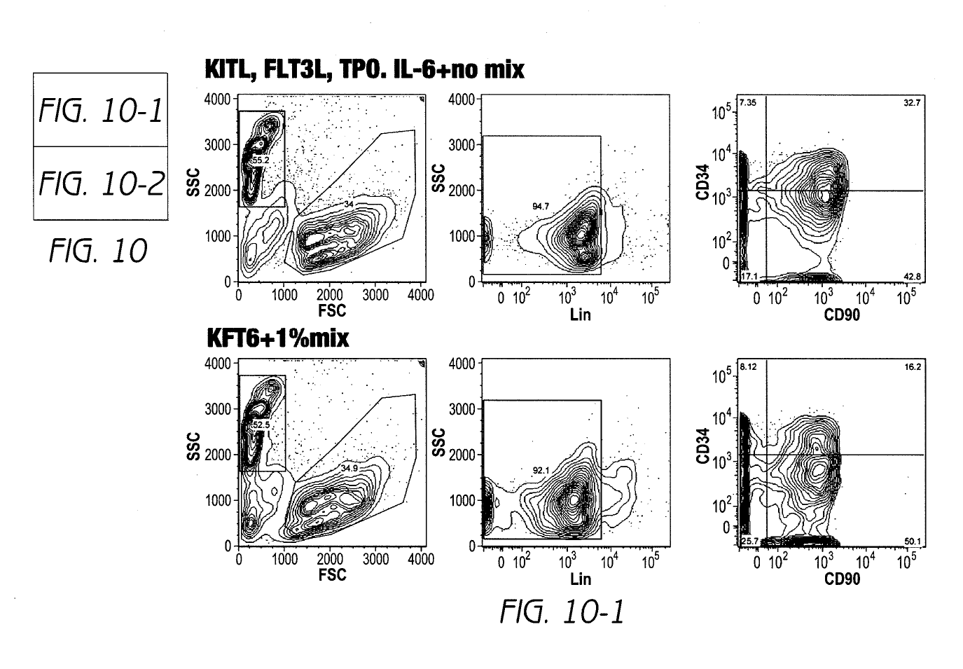

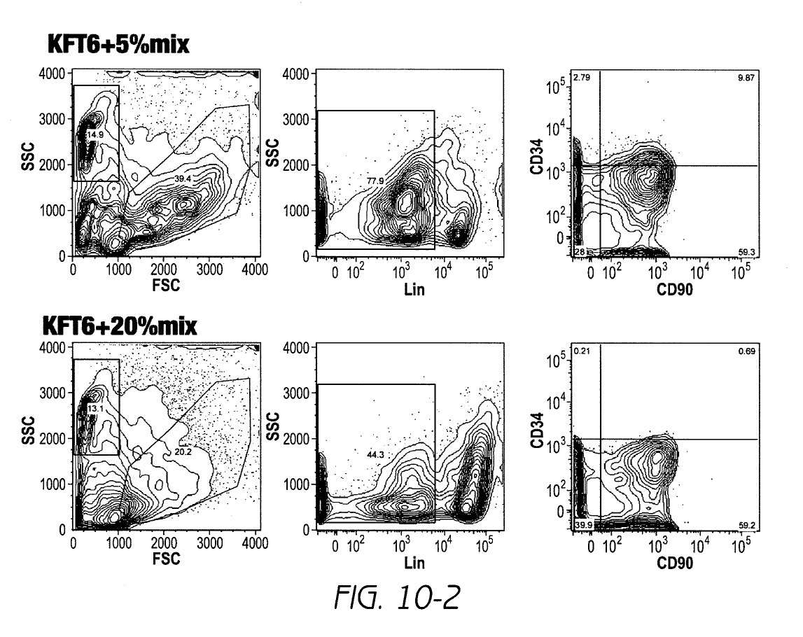

FIGS. 7, 8, 9 and 10 show examples of the day 8 flow analysis.

| | | Cells | Lin | |
|---|---|---|---|---|
| Group | Day | present | neg (%) | CD34+ |
| KITL, TPO, FLT3L | 5 | 1,752 | 96% | 794 |
| KITL, TPO, FLT3L | 5 | 1,876 | 99% | 1,119 |
| KITL, TPO, FLT3L + IL-3 | 5 | 4,117 | 93% | 1,438 |
| KITL, TPO, FLT3L + IL-6 | 5 | 3,174 | 96% | 1,703 |
| KITL, TPO, FLT3L + 1% PL | 5 | 2,150 | 88% | 935 |
| KITL, TPO, FLT3L + 5% PL | 5 | 4,329 | 73% | 1,165 |
| KITL, TPO, FLT3L + 20% PL | 5 | 1,125 | 70% | 26 |
| KITL, TPO, FLT3L | 8 | 9,328 | 97% | 1,087 |
| KITL, TPO, FLT3L | 8 | 8,088 | 98% | 841 |
| KITL, TPO, FLT3L + IL-3 | 8 | 59,344 | 68% | 8,737 |
| KITL, TPO, FLT3L + IL-3 | 8 | 57,483 | 72% | 7,902 |
| KITL, TPO, FLT3L + IL-6 | 8 | 15,102 | 98% | 1,582 |
| KITL, TPO, FLT3L + IL-6 | 8 | 12,736 | 97% | 1,306 |
| No GF + 0% PL | 8 | 97 | 55% | 15 |
| No GF + 1% PL | 8 | 1,944 | 77% | 64 |
| No GF + 5% PL | 8 | 16,874 | 42% | 75 |
| KITL, TPO, FLT3L + IL-6 + | 8 | 19,437 | 95% | 7,372 |
| 0% PL | | | | |
| KITL, TPO, FLT3L + IL-6 + | 8 | 21,148 | 92% | 4,737 |
| 1% PL | | | | |
| KITL, TPO, FLT3L + IL-6 + | 8 | 128,174 | 78% | 12,641 |
| 5% PL | | | | |
| KITL, TPO, FLT3L + IL-6 + | 8 | 88,587 | 44% | 353 |
| 20% PL | | | | |

The data in the tables above are derived from the flow cytometric analysis, using beads (visible in the upper left of the scatter plots in FIGS. 6, 8 and 9) to obtain absolute cell counts. The cell counts in wells containing high concentration of PL are inaccurate due to the inability to separate debris (red cells) from the lysate from the cells during analysis.

The above experiments show that at concentrations of about 5% and higher of the platelet lysate can act as a potent co-stimulator, increasing the number of cells in culture by 5 to 10 fold over those seen by stimulation with specific recombinantly produced growth factors alone.

The platelet lysate may induce more rapid differentiation, noted as increased percentages of Lie^{pos} cells as well as decreases in CD34^{pos} cells. The number of CD34^{pos} cells is especially low in cultures only stimulated by the platelet lysate.

FIG. 7 shows that the addition of the platelet releasate shifts the progenitor population from mixed CMP/GMP to CMP.

The use of the platelet lysate as a co-stimulator can result in an absolute, but not relative, increase in the number of CD34^{pos} cells. However, the absolute numbers are not much higher in the 4 growth factor+platelet lysate combination than in some of the 4 growth factor combinations.

These conclusions are limited by the fact that these analyses are based on (i) single wells containing the cells from (ii) a single individual and using the platelet lysate from (iii) a single individual. The platelets and the cells in these experiments did not come from the same individual. Furthermore (iv) there currently is no functional confirmation of the actual potential of the cells defined by phenotype.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of culturing cells, comprising the steps of:
extracting blood from a patient;
separating platelets and plasma from the blood;
forming a platelet-rich plasma by suspending the platelets in the plasma at a concentration higher than found in the blood;
adding the platelet-rich plasma to a cell culture medium;
placing cells on the culture medium; and
proliferating the cells on the culture media.

2. The method of claim 1, further comprising:
packaging the proliferated cells.

3. The method of claim 1, wherein the blood and the cells are obtained from the same patient.

4. The method of claim 1, wherein the cells are fibroblasts.

5. The method of claim 1, wherein the platelet-rich plasma is not activated by addition of exogenous activators.
